(19) United States
(12) Patent Application Publication
(10) Pub. No.: US 2026/0338528 A1
(43) Pub. Date: Oct. 29, 2026

(54) TURF HEIGHT AND SOIL TILTH INDICATOR

(71) Applicant: K Bar W Company, Shawnee, OK (US)

(72) Inventor: Christopher S. Kluding, Shawnee, OK (US)

(21) Appl. No.: 19/394,319

(22) Filed: Nov. 19, 2025

Related U.S. Application Data
(60) Provisional application No. 63/833,377, filed on Nov. 21, 2024.

(51) Int. Cl. G01B 5/06; A63B 57/50; G01N 33/24

(52) U.S. Cl. CPC G01B 5/061; A63B 57/50; G01N 33/24

(57) ABSTRACT

A multi-purpose tool for lawn care is disclosed, primarily configured for indicating turf height and soil tilth. The tool comprises a handle coupled to a rod having a beveled tip for ground insertion. A removable cap fits onto the rod and includes markings for measuring turf height. The rod is color-indexed to indicate soil tilth when the cap is removed and the rod is inserted into the ground. A corresponding method involves using the tool with the cap in place to measure turf height against the cap markings, and removing the cap to expose the color-indexed rod for ground insertion and observation of soil tilth. The handle may be shaped as a golf putter head, and the grip can be shaped as a golf club grip.

### CROSS-REFERENCE TO RELATED APPLICATIONS

This application claims the benefit of U.S. Provisional Application No. 63/833,377, filed Nov. 21, 2024, which is hereby incorporated herein by reference in its entirety.

### BACKGROUND

Maintaining healthy and aesthetically pleasing lawns, golf courses, and other turf areas requires careful and integrated management of several environmental and physical factors. The most critical of these factors include turf height and the underlying soil conditions, which are often characterized by its soil tilth (or compaction). Soil tilth is a comprehensive measure of soil quality that reflects its physical state, including its structure, porosity, and consistency, and is intrinsically linked to the soil's ability to hold and transmit water. Both turf height and soil tilth significantly and interdependently impact the health, growth, and appearance of turf, with poor tilth-whether due to excessive compaction or adverse water conditions-often being the primary limiting factor for root development and nutrient uptake.

Currently, professional landscapers, golf course superintendents, and home lawn enthusiasts often rely on separate and distinct tools to perform these two essential assessments. For instance, specialized gauges are used to measure the height of grass blades. At the same time, other implements, such as probes or penetrometers, are employed to determine soil compaction or the ease with which a root system can develop. The need to carry and utilize multiple tools for these routine maintenance tasks can be inconvenient, time-consuming, and may lead to less frequent or less thorough assessments. There remains a need for an improved, more integrated, and efficient device for simultaneously or sequentially performing these familiar turf and soil health evaluations.

The present invention relates to a multi-purpose tool for lawn and turf care. Specifically, this tool addresses the need for a convenient and accurate method to measure both turf height and soil tilth (which encompasses physical properties such as compaction and moisture content) in a lawn or golf course setting. Existing tools often require separate devices for these tasks, which can be cumbersome and inefficient. This invention provides a single, easy-to-use tool that combines these functions in a compact and ergonomic design.

### SUMMARY OF THE INVENTIVE CONCEPTS

The inventive concepts disclosed herein are directed to a turf height and soil tilth indicator. The indicator comprises a handle, a rod, and a removable cap. The handle enables users to hold and operate the device comfortably. In one illustrative embodiment, the handle is shaped like a golf putter head. The rod is fixed to the handle and has a beveled tip on the opposite end for ground insertion. The removable cap slides onto the shaft and contains markings for measuring turf height. The rod is color-indexed with different zones to indicate soil tilth.

### BRIEF DESCRIPTION OF THE DRAWINGS

To assist those of ordinary skill in the relevant art in making and using the inventive concepts disclosed herein, reference is made to the appended drawings and schematics, which are not intended to be drawn to scale and in which like reference numerals are intended to refer to the same or similar elements for consistency. For purposes of clarity, not every component may be labeled in every drawing. Certain features and views of the figures may be shown exaggerated and not to scale or schematic in the interest of clarity and conciseness. In the drawings:

FIG. 1 is a perspective view of a turf height and soil tilth indicator constructed in accordance with the inventive concepts disclosed herein.

FIG. 2 is a perspective view of the turf height and soil tilth indicator of FIG. 1 shown with a cap removed to expose a tip of the rod.

FIG. 3 is an enlarged perspective view of the tip of FIG. 2.

FIG. 4 is an in-use view illustrating the turf height and soil tilth indicator being used to measure turf height.

FIG. 5 is an in-use view illustrating the turf height and soil tilth indicator being used as a soil tilth indicator.

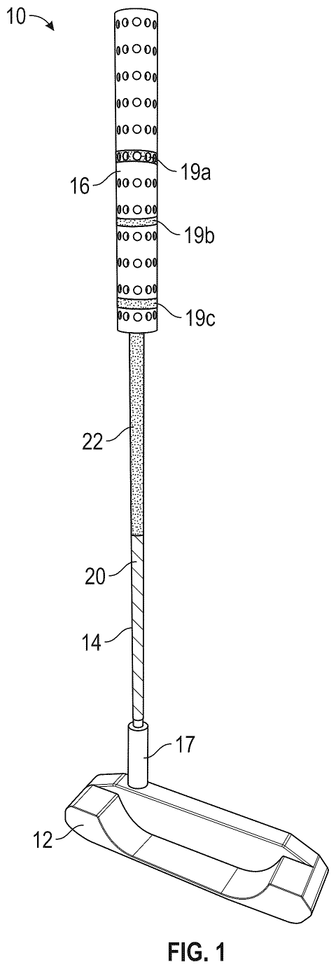

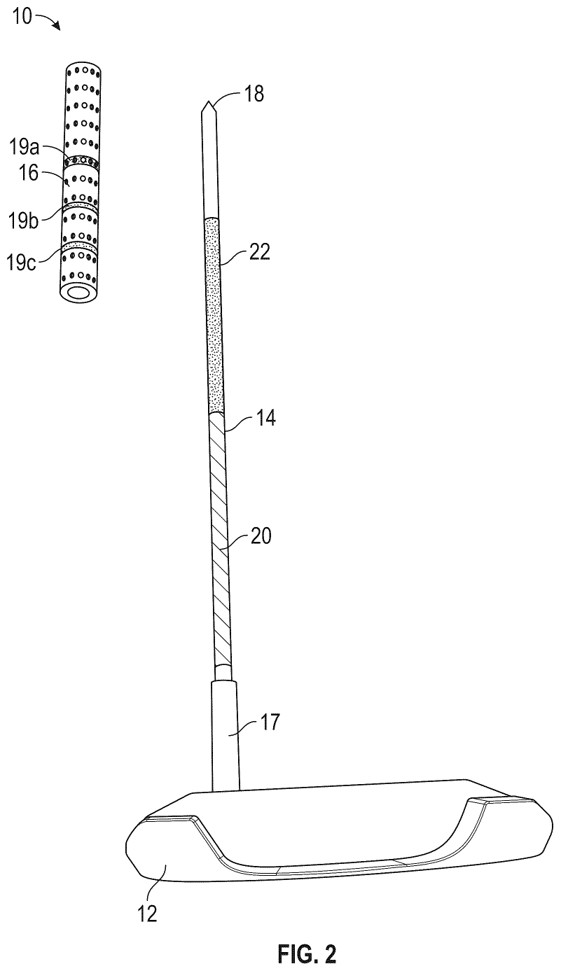

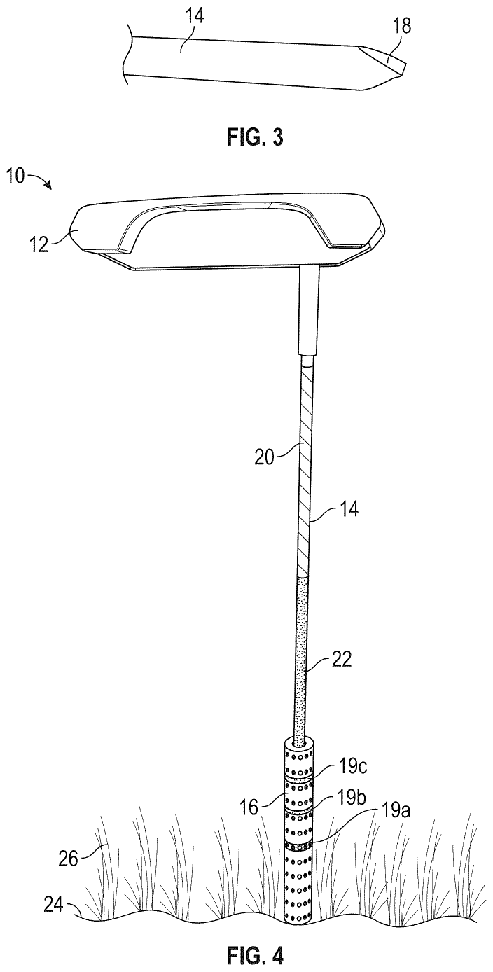

### DETAILED DESCRIPTION OF EXEMPLARY EMBODIMENTS

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) are capable of other embodiments or being practiced or carried out in various ways. The language used herein is intended to be given the broadest possible scope and meaning; the embodiments are meant to be exemplary, not exhaustive. Also, it is to be understood that the phraseology and terminology employed herein are for the purpose of description and should not be regarded as limiting.

Unless otherwise defined, scientific and technical terms used in connection with the presently disclosed and claimed inventive concept(s) shall have the meanings commonly understood by those of ordinary skill in the art. Further, unless otherwise required by context, singular terms shall include pluralities, and plural terms shall include the singular.

Given the present disclosure, all the articles, compositions, and/or methods disclosed and claimed herein can be made and executed without undue experimentation. While the articles, compositions, and methods of the inventive concept(s) have been described in terms of preferred embodiments, it will be apparent to those of skill in the art that variations may be applied to the articles, compositions and/or methods and in the steps or in the sequence of steps of the methods described herein without departing from the concept, spirit and scope of the inventive concept(s). All similar substitutes and modifications apparent to those skilled in the art are deemed to be within the spirit, scope, and concept of the inventive concept(s) as defined by the appended claims.

As utilized under the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” indicates that a value includes the inherent variation of error for the device, the method employed to determine the value or the variation among the study subjects.

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if the order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that, typically, there is no limit on the number of items or terms in any combination unless otherwise apparent from the context.

In the following detailed description of embodiments of the inventive concept, numerous specific details are set forth in order to provide a more thorough understanding of the inventive concept. However, it will be apparent to one of ordinary skill in the art that the inventive concept within the disclosure may be practiced without these specific details. In other instances, well-known features have not been described in detail to avoid unnecessarily complicating the instant disclosure.

Finally, as used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Referring to the provided drawings, a turf height and soil tilth indicator 10 (hereafter the indicator 10), constructed in accordance with the inventive concepts disclosed herein, is illustrated. The indicator 10 is generally used for lawn and turf care. Broadly, the indicator 10 includes a handle 12, a rod or shaft 14, and a cap 16.

The handle 12 is designed to be grasped by an operator for ease of use. While an illustrative embodiment of the handle 12 is shaped like a golf putter head for ergonomic appeal and to leverage a familiar form factor, it will be appreciated that the shape of the handle 12 is not limited to this design. Alternative handle shapes are contemplated, including but not limited to T-shaped handles, pistol grips, or simple cylindrical grips.

The handle 12 includes an integrated collar or socket 17 to accept the rod 14. The handle 12 may be fabricated from a variety of materials, including those compatible with 3D printing, as well as molded plastics, machined metals, or wood. For marketing and branding purposes, the handle 12 can be produced in a wide variety of colors, which can be effective for building brand recognition and strengthening customer engagement.

The rod 14 is attached to the handle 12. As shown in an illustrative embodiment, the rod 14 is a 3/16 inch OD steel rod, approximately 12 inches in length. However, the rod 14 can be fabricated from alternative materials such as carbon fiber, fiberglass, or a durable plastic polymer, provided the material is rigid enough to be inserted into soil. One end of the rod 14 is permanently fixed to the handle 12. The opposite end of the rod 14 is a beveled tip 18, which is configured for ground insertion.

The cap 16 may be configured to friction-fit onto the rod 14. While the cap 16 can be configured to resemble a grip of a golf putter, its shape is not limited to this design and other configurations are contemplated. The cap 16 is marked with specific turf height measurements 19a, 19b, and 19c, such as at 2″, 3″, and 4″.

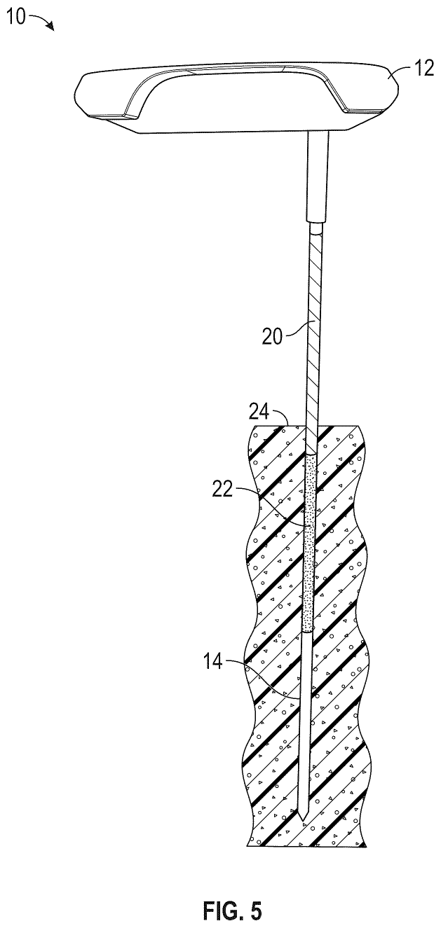

The dual use of the turf height and soil tilth indicator 10 is accomplished by either keeping the cap 16 on for turf height measurement (as illustrated in FIG. 4) or removing it for soil tilth assessment (as illustrated in FIG. 5). These markings 19a-19c allow the user to measure turf height by placing the end of the cap 16 against the ground 24 and comparing the height of the grass 26 to the markings, as depicted in FIG. 4.

A portion of the rod 14 is colored or marked to serve as a soil tilth indicator (or compaction). The rod 14 is color-indexed with different zones to provide an immediate visual assessment. A first zone 20 (for example, colored green) is designated to indicate good soil tilth. Good soil tilth means that soil penetration is possible without undue force. A second zone 22 (for example, colored orange) is used to indicate poor soil tilth, signifying an area where soil penetration is limited due to compaction. By inserting the beveled tip of the rod into the ground, the operator can determine the depth of penetration, and the colors observed on the rod relative to the soil surface correspond to the soil tilth. However, alternative methods for indicating soil tilth are also contemplated, such as different color schemes, patterns, or simple scales of engraved lines.

Poor soil tilth, often resulting from compaction, hinders root growth and turf health. The most direct mechanical solution to break up this compaction is through aeration, which creates channels for air, water, and nutrients to penetrate the root zone. This can be accomplished through core aeration, where small plugs of soil are removed, or, for milder issues, spike aeration may be employed. To durably improve soil structure and prevent rapid re-compaction, one should focus on amending the soil by applying organic matter, such as compost, to the turf surface, allowing it to work its way into the soil to act as a conditioner. Finally, adopting better cultural practices is essential, which includes controlling traffic on the turf, especially when the soil is wet, and ensuring proper watering techniques. Deep, infrequent watering is preferable, as it encourages the development of deeper, healthier root systems, which naturally help “till” the soil and make the turf more resilient to compaction stress.

From the above description, it is clear that the inventive concept(s) disclosed herein are well adapted to carry out the objects and to attain the advantages mentioned herein, as well as those inherent in the inventive concept disclosed herein. While exemplary embodiments of the inventive concept disclosed herein have been described for purposes of this disclosure, it will be understood that numerous changes may be made that will readily suggest themselves to those skilled in the art and which are accomplished without departing from the scope of the inventive concepts disclosed and claimed herein.

1. A turf height and soil tilth indicator for lawn care, comprising:
a handle;
a rod attached to the handle, having a beveled tip on an end opposite the handle; and
a removable cap configured to friction fit onto the rod, the cap having markings to indicate turf height,
wherein the rod is color-indexed to indicate soil tilth when the grip cap is removed.

2. The indicator of claim 1, wherein the handle is shaped as a golf putter head.

3. The indicator of claim 1, wherein the cap is shaped as a grip of a golf putter.

4. The indicator of claim 1, wherein the rod is approximately 12 inches in length.

5. The indicator of claim 1, wherein the rod has a diameter of approximately 3/16 inch.

6. The indicator of claim 1, wherein the markings on the grip cap include measurements at 2 inches, 3 inches, and 4 inches.

7. The indicator of claim 1, wherein the color-indexed rod includes a green zone for good soil tilth and an orange zone for poor soil tilth.

8. A method for indicating turf height and soil tilth, comprising:
providing a tool with a handle, a rod, and a removable cap with markings;
using the tool with the cap in place to measure turf height by comparing the turf to the markings on the cap; and
removing the cap to expose a beveled tip on the rod, inserting the beveled tip of the rod into a surface of a soil until a resistance is felt, and observing the color-indexed portion of the rod relative to the surface of the soil to determine the depth of penetration.
